(19) United States
(12) Patent Application Publication
(10) Pub. No.: US 2010/0209460 A1
(43) Pub. Date: Aug. 19, 2010

(54) ANTI-ODOR AGENT HAVING DISINFECTING OR BACTERICIDAL EFFECT

(75) Inventor: Wolf-Ruediger Pietsch, Hoevelhof (DE)

Correspondence Address: Brinks Hofer Gilson & Lione/Ann Arbor, 524 South Main Street, Suite 200, Ann Arbor, MI, 48104, US

(73) Assignee: Tacco Footcare KG, Bad Wuennenberg (DE)

(21) Appl. No.: 12/702,646

(22) Filed: Feb. 9, 2010

Related U.S. Application Data
(60) Provisional application No. 61/151,534, filed on Feb. 11, 2009.

(51) Int. Cl. A61K 33/34; A61K 9/00; A61P 43/00; A61L 9/01

(52) U.S. Cl. 424/400; 424/76.21; 424/76.1; 977/773

(57) ABSTRACT

A liquid-based anti-odor agent is provided that has a disinfectant or bactericidal effect for the interiors of shoes and for shoe parts, particularly for shoe insoles, characterized in that the anti-odor agent comprises copper or copper compounds.

### CROSS-REFERENCE TO RELATED APPLICATIONS

This application claims the benefit of provisional application Ser. No. 61/151,534, filed on Feb. 11, 2009, the contents of which are incorporated herein by reference in their entirety.

### FIELD

The present disclosure relates to a liquid-based anti-odor agent for the interiors of shoes and for shoe parts, particularly for shoe insoles.

### BACKGROUND

The statements in this section merely provide background information related to the present disclosure and may not constitute prior art.

Anti-odor agents are typically presented to the end user as sprayable products. The primary object of such anti-odor agents is to bind odors and counteract bacterial growth by a disinfectant or bactericidal effect. For this purpose, appropriate disinfectants or common toxins may be used in quantities and concentrations that are safe for humans.

### SUMMARY

The present disclosure provides an odor agent which meets requirements with respect to the disinfectant or bactericidal effect thereof, and at the same time is relatively safe to human health.

This is attained according to the present disclosure by an anti-odor agent of the type mentioned above, which comprises copper or copper compounds.

According to the present disclosure, it was found that copper has a very effective disinfectant and bactericidal effect for the application in question. Copper is toxic for many microorganisms at very low concentrations, which are safe for vertebrates and humans. In other words, a person may consume about 0.04 g of copper per day without suffering any damage to his health.

In a refinement of the present disclosure, the anti-odor agent may be applied as an aerosol.

According to one form of the present disclosure, copper or copper compounds are present in the anti-odor agent in the form of nanoscale particles having a dimension of about<150 nm.

Copper or copper compounds may, however, also be present in dissolved form in the anti-odor agent. In addition to copper or copper compounds, a second component acting as a carrier for the copper/copper compound is provide, and is generally able to evaporate. This second component may be, for example, ethanol, propane, butane or isopropanol. Such chemicals are commonly used as carriers in similar applications, such as sprays for improving the air quality in bathrooms, and sprays for the impregnation of clothes or shoes or hair spray, among others.

Another subject matter of the present disclosure is a pump spray device that can be manually actuated, particularly as a spray can, comprising an anti-odor agent of the type mentioned in claim 1.

A further subject matter of the present disclosure is the use of an anti-odor agent comprising copper or copper compounds for the care of the interiors of shoes or of shoe parts, particularly of shoe insoles.

Further areas of applicability will become apparent from the description provided herein. It should be understood that the description and specific examples are intended for purposes of illustration only and are not intended to limit the scope of the present disclosure.

### DETAILED DESCRIPTION

The following description is merely exemplary in nature and is not intended to limit the present disclosure, application, or uses.

A liquid-based anti-odor agent having a disinfectant or bactericidal effect for the interiors of shoes and for shoe parts is provided by the present disclosure. The agent is provided particularly for shoe insoles, characterized in that the anti-odor agent comprises copper or copper compounds.

In one form, the anti-odor agent can be applied as an aerosol. In another form, the copper or copper compounds are present in the form of nanoscale particles having a dimension of about<150 nm. In still another form, the copper or copper compounds are present in dissolved form.

In another form, a pump spray device that can be manually actuated is provided, and particularly as a spray can that comprises the anti-odor agent.

According to a method of the present disclosure, the use of an anti-odor agent comprising copper or copper compounds for the care of the interiors of shoes or of shoe parts is provided, and particularly for shoe insoles.

It should be noted that the disclosure is not limited to the various forms described and illustrated as examples. A large variety of modifications have been described and more are part of the knowledge of the person skilled in the art. These and further modifications as well as any replacement by technical equivalents may be added to the description, without leaving the scope of the protection of the disclosure and of the present patent.

What is claimed is:

1. A liquid-based anti-odor agent having at least one of a disinfectant and bactericidal effect for the interiors of shoes and for shoe parts, particularly for shoe insoles, characterized in that the anti-odor agent comprises at least one of copper and copper compounds.

2. The anti-odor agent according to claim 1, characterized in that the agent can be applied as an aerosol.

3. The anti-odor agent according to claim 1, characterized in that the copper and copper compounds are present in the form of nanoscale particles having a dimension of about<150 nm.

4. The anti-odor agent according to claim 1, characterized in that the copper and copper compounds are present in dissolved form.

5. A pump spray device that can be manually actuated, particularly as a spray can, comprising the anti-odor agent according to claim 1.

6. Use of an anti-odor agent comprising at least one of copper and copper compounds for the care of interiors of shoes or of shoe parts, particularly shoe insoles.
